(19) United States
(12) Patent Application Publication
(10) Pub. No.: US 2010/0120843 A1
(43) Pub. Date: May 13, 2010

(54) PHARMACEUTICAL COMPOSITION FOR TREATING ALCOHOL-INDUCED LIVER INJURY COMPRISING (4S,5S)-5-FLUOROMETHYL-5-HYDROXY-4-(AMINO)-DIHYDROFURAN-2-ONE OR PHARMACEUTICALLY ACCEPTABLE SALT THEREOF

(75) Inventors: Hye Kyung CHANG, Daejeon (KR); Yeong Soo OH, Daejeon (KR); Hyun Ik SHIN, Daejeon (KR); Mi Jeong PARK, Daejeon (KR); Jung Gyu PARK, Daejeon (KR); Yong Han PAIK, Seoul (KR)

Correspondence Address: BIRCH STEWART KOLASCH & BIRCH, PO BOX 747, FALLS CHURCH, VA, 22040-0747, US

(73) Assignee: LG Life Sciences Ltd., Seoul (KR)

(21) Appl. No.: 12/270,592

(22) Filed: Nov. 13, 2008

(51) Int. Cl. A61K 31/4725

(52) U.S. Cl. 514/307

(57) ABSTRACT

The present invention relates to a pharmaceutical composition for treating alcohol-induced liver injury comprising (4S,5S)-5-fluoromethyl-5-hydroxy-4-({[(5R)-5-isopropyl-3-(isoquinolin-1-yl)-4,5-dihydro-5-isoxazolyl]carbonyl}amino)-dihydrofuran-2-one or pharmaceutically acceptable salt thereof, and a use thereof.

### FIELD OF THE INVENTION

The present invention relates to a pharmaceutical composition for treating alcohol-induced liver injury comprising (4S,5S)-5-fluoromethyl-5-hydroxy-4-({[(5R)-5-isopropyl-3-(isoquinolin-1-yl)-4,5-dihydro-5-isoxazolyl]carbonyl}amino)-dihydrofuran-2-one or pharmaceutically acceptable salt thereof, and a use thereof.

### BACKGROUND ART

It is known that the continual alcohol ingestion causes fatty liver and subsequent alcoholic steatohepatitis. Alcoholic liver diseases had been known to be caused by nutritional deficiency for the last several decades. But, at present, alcohol itself is regarded as a toxic substance to liver. About 15% of alcoholics suffer from progression into alcoholic cirrhosis, and most of them die of hepatic failure or complications of liver cirrhosis. Alcoholic liver diseases can be divided into the following 3 classes of diseases according to pathohistology: fatty liver, alcoholic hepatitis, and alcoholic cirrhosis.

Although more and more evidences for association of chronic inflammation in alcohol-induced liver injury have been shown, definite mode of action for alcohol-induced liver injury is not well-known and yet to be defined. In an individual of alcohol dependence, either immunological response to antigen originated from oxidative stress or lipopolysaccharide (LPS) from large intestine has been reported as the cause of liver injury (Nagata K, Suzuki H, Sakaguchi S; Common pathogenic mechanism in development progression of liver injury caused by non-alcoholic or alcoholic steatohepatitis; J Toxicol Sci.; 2007 December; 32(5):453-468). Differentiated from viral hepatitis and autoimmune disease associated hepatitis, causative agents of immunological response mediated by denatured protein due to oxidative stress have not been clearly identified, and yet hepatitis is known to be accelerated by synergic interaction with immunological response mediated by elevation of intestinal LPS permeability due to alcohol ingestion. An anti-oxidant such as silymarin is used to alleviate the symptom of such hepatitis, but its treating effect is uncertain.

Steroids are usually used to treat severe alcoholic hepatitis, but the steroid treatment may cause systemic infections resulting in serious conditions. Moreover, currently available steroid therapies are still ineffective in about 40% of patients with alcoholic hepatitis.

### SUMMARY OF THE INVENTION

The purpose of the present invention is to provide a pharmaceutical composition for effective treatment of alcohol-induced liver injury.

To meet the above purpose, the present invention provides a use of (4S,5S)-5-fluoromethyl-5-hydroxy-4-({[(5R)-5-isopropyl-3-(isoquinolin-1-yl)-4,5-dihydro-5-isoxazolyl]carbonyl}amino)-dihydrofuran-2-one of the following Formula 1 (referred to as CF1 (Compound of Formula 1) hereinafter) or pharmaceutically acceptable salt thereof for treating alcohol-induced liver injury.

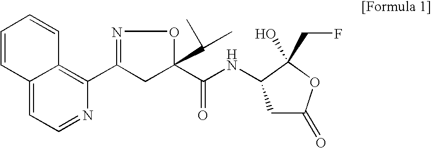

### BRIEF DESCRIPTION OF THE FIGURES

FIG. 1 is a graphical presentation of fluorometric assay results of caspase-3 activity in experimental groups of control diet (Control), control diet with CF1 (Control+CF1), EtOH diet (EtOH), and EtOH diet with CF1 (EtOH+CF1).

FIG. 2 is a graphical presentation of fluorometric assay results of caspase-8 activity in experimental groups of control diet (Control), control diet with CF1 (Control+CF1), EtOH diet (EtOH), and EtOH diet with CF1 (EtCO+CF1).

FIG. 3 is a graphical presentation showing effect of CF1 on serum aspartate transaminase (AST) and alanine transaminase (ALT) values in experimental groups of control diet (Control), control diet with CF1 (Control+CF1), EtOH diet (EtOH), and EtOH diet with CF1 (EtOH+CF1).

FIG. 4A is a set of representative photomicrographs of liver after hematoxylin and eosin (H&E) staining at 100 times magnification, and FIG. 4B is a set of representative photomicrographs of liver section processed for Masson's trichrome at 100 times magnification.

FIG. 5 is a set of representative photomicrographs of liver after terminal deoxynucleotidyl transferase-mediated deoxyuridine triphosphate nickend labeling (TUNEL) staining at 400 times magnification.

FIG. 6 is a graphical presentation of quantitative results by number of TUNEL positive cells per field in experimental groups of control diet (Control), control diet with CF1 (Control+CF1), EtOH diet (EtOH), and EtOH diet with CF1 (EtOH+CF1).

### DETAILED DESCRIPTION OF THE INVENTION

The present invention is described in detail hereafter.

The present invention relates to a use of (4S,5S)-5-fluoromethyl-5-hydroxy-4-({[(5R)-5-isopropyl-3-(isoquinolin-1-yl)-4,5-dihydro-5-isoxazolyl]carbonyl}amino)-dihydrofuran-2-one of the following Formula 1 or pharmaceutically acceptable salt thereof for treating alcohol-induced liver injury.

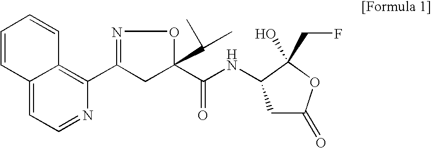

According to one embodiment of the present invention, a pharmaceutical composition for treating alcohol-induced liver injury comprising CF1 or a pharmaceutically acceptable salt thereof is provided.

According to another embodiment of the present invention, a method for manufacturing of pharmaceutical composition for treating alcohol-induced liver injury comprising admixing CF1 or a pharmaceutically acceptable salt thereof with pharmaceutically acceptable carrier is provided.

According to still another embodiment of the present invention, a use of CF1 or a pharmaceutically acceptable salt thereof for manufacturing a pharmaceutical composition for treating alcohol-induced liver injury is provided.

According to still another embodiment of the present invention, a method for treating alcohol-induced liver injury, comprising administrating an effective amount of the pharmaceutical composition comprising CF1 or a pharmaceutically acceptable salt thereof to a patient suffering from alcohol-induced liver injury is provided.

According to still another embodiment of the present invention, a use of the pharmaceutical composition comprising CF1 or a pharmaceutically acceptable salt thereof for treating alcohol-induced liver injury is provided.

The manufacturing method of CF1, the manufacturing method of intermediate of CF1 and use of CR1 for treating hepatic diseases by hepatitis virus, acute hepatitis and hepatic cirrhosis are disclosed in International Publication No. WO 2006/090997.

In the present invention, the alcoholic liver diseases include fatty liver, alcoholic hepatitis, alcoholic cirrhosis and the like.

The present inventors have discovered the role of caspase in alcohol-induced liver injury and that alcohol-induced liver injury call be efficiently treated by inhibiting the caspase activity.

CF1 of the present invention is usually synthesized from the mixture of the compound of following Formula 6 and the compound of the following Formula 7.

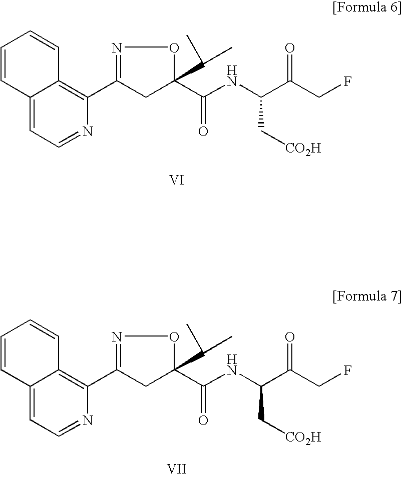

In solution phase, CF1 and the compound of Formula 6 exist in equilibrium as follows:

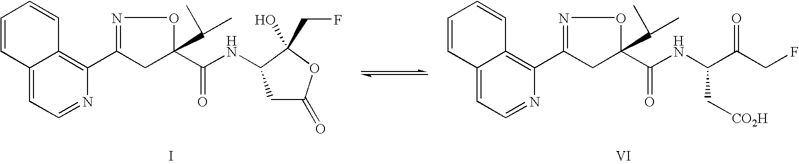

The equilibrium state can be verified by solution NMR (nuclear magnetic resonance), and the structure of CF1 was determined by X-ray crystallography and solid NMR. CF1 can be obtained in both amorphous form and crystalline form, and the crystalline form is more stable. CF1 can be obtained in the crystalline form exclusively by crystallization method.

According to the purpose, CF1 of the present invention may be formulated into various pharmaceutical forms for administration. To prepare the pharmaceutical composition according to the present invention, an effective amount of CF1 is mixed with a pharmaceutically acceptable carrier that may be selected in various forms depending on the formulation to be prepared.

CF₁ of the present invention may be formulated as a parenteral injection, or percutaneous or oral preparation depending on its application purpose. It is preferable to formulate the composition in a unit dosage form in terms of administration easiness and dose uniformity.

For the oral preparation, any conventional pharmaceutical carrier may be used. For example, water, glycols, oils, alcohols and the like may be used for oral liquid preparations such as suspensions, syrups, elixirs and solutions. Starches, sugars, kaolin, lubricants, binders, disintegration agents and the lie may be used for solid preparations such as powders, pills, capsules and tablets. Tablets and capsules are the most convenient dosage unit forms for their administration easiness. Tablets and pills are preferably formulated as enteric-coated preparation.

For the parenteral preparation, sterile water is usually used as the carrier, and other ingredients such as solubility aids may be further used. Injections, for example, sterilized aqueous or oily suspension for injection, can be prepared according to known procedures using suitable dispersing agent, wetting agent, or suspending agent. Solvents that can be used for preparing injections include water, Ringer's fluid, and isotonic NaCl solution, and sterilized fixing oil may also be used conveniently as the solvent or suspending media. Any non-stimulative fixing oil including mono-, di-glyceride may be used for this purpose. Fatty acid such as oleic acid may also be used for injections.

For the percutaneous preparation, the carrier may include a penetration enhancing agent and/or a suitable wetting agent, optionally in combination with suitable additives with no significant irritation to skin. For additives, those enhancing the administration through the skin and/or assisting the preparation of a desired composition are selected. These percutaneous preparations are administered via various routes, e.g., a transdermal patch, a spot-on, or an ointment.

When CF1 is used for clinical purpose, it is preferably administered to the subject patient in a total amount ranging from 0.1 to 100 mg per kg of body weight a day via either single dosage or divided dosage. Specific administration dosage for a specific individual patient can vary depending on specific compound to be used, body weight, sex, hygienic condition and diet of the subject patient, time and method of administration, excretion rate, mixing ratio of agent, severity of disease to be treated, etc.

The present invention is explained in more detail by the following Examples, but is not limited thereto.

### PREPARATORY EXPERIMENT

### Synthesis of CF1

CF1 of the present invention was synthesized through the following Reaction Scheme 1.

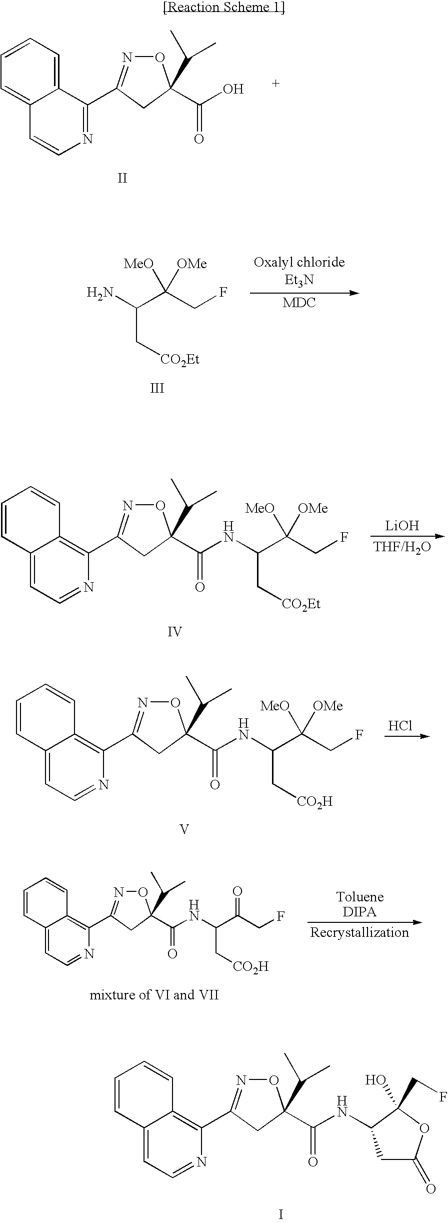

### Preparation Example 1

5-fluoro-3-[((R)-S-isopropyl-3-(isoquinolin-1-yl)-4,5-dihydro-isoxazole-5-carbonyl)-amino]-4,4-dimethoxy-pentanoic acid ethyl ester (Compound IV)

15.5 g of the compound of Formula II (Compound II, 54.5 mmol) was dissolved in 150 mL of methylene chloride (MDC), and the temperature was adjusted to 0° C., and then 7.1 mL (81.7 mmol) of oxalyl chloride and 0.2 mL (2.6 mmol) of dimethyl formamide (DMF) were added thereto with maintaining the inner temperature below 12° C. The reaction mixture was stirred at 20° C. for about 2 hours, and concentrated under reduced pressure. The concentrated reaction mixture was diluted with 150 mL of methylene chloride (MDC), then the temperature was adjusted to 0° C., and 16.5 g of triethylamine (Et₃N) was added thereto, and a solution of 12.8 g of the compound of Formula III (Compound III, 57.4 mmol) dissolved in 30 mL of methylene chloride was slowly added thereto over 20 minutes. The resulting reaction mixture was stirred at 25° C. for 1.5 hours, and then a mixed solution of 120 mL of 10% sodium hydrogen carbonate aqueous solution and 60 mL of 1 N sodium hydroxide aqueous solution was added thereto to terminate the reaction. After the separation of organic layer, aqueous layer was extracted with methylene chloride (150 mL×3). The combined organic layer was concentrated under reduced pressure to give the title compound (Compound IV; 30.1 g, quantitative yield). This compound was used in the next step without further purification.

¹H NMR (500 MHz, CDCl₃): 9.12 (q, 1H), 8.53 (m, 1H), 7.85-7.25 (m, 4H), 4.80 (m, 1H), 4.54-4.34 (m, 2H), 4.14 (q, J=7.4 Hz, 2H), 3.99 (2d, J=18.4 Hz, 1H), 3.81 (m, 1H), 3.78 (2d, J=8.6 Hz, 1H), 3.33 (d, 3H), 3.20 (d, 3H), 2.75 (m, 3H), 2.53 (m, 1H), 2.39 (heptet, J=6.7 Hz, 1H), 1.27 (t, J=7.4 Hz, 1.5H), 1.07 (m, 6H), 0.97 (t, J=7.4 Hz, 1.5H)

### Preparation Example 2

### 5-Fluoro-3-[((R)-5-isopropyl-3-(isoquinolin-1-yl)-4,5-dihydro-isoxazole-5-carbonyl)-amino]-4,4-dimethoxy-pentanoic acid (Compound V)

30.1 g of the compound obtained from the above Preparation Example 1 (Compound IV, 61.6 mmol), together with 7.76 g (185 mmol) of lithium hydroxide monohydrate, was added to a mixed solvent of 168 mL of tetrahydrofuran (THF) and 42 mL of water, and stirred at about 40° C. for 4 hours. The reaction mixture was concentrated under reduced pressure to remove tetrahydrofuran in the solvent. 180 mL of 1 N sodium hydroxide aqueous solution was added thereto, and the mixture was washed with toluene (120 mL×2). The aqueous layer was acidified with 66 mL of 6 N hydrochloric acid aqueous solution, and extracted with methylene chloride (150 mL×3), and the combined organic layer was concentrated under reduced pressure to give the title compound (Compound V, 25.4 g, 89%). This compound was used in the next step without further purification.

¹H NMR (400 MHz, CDCl₃): 9.10-8.92 (m, 1H), 8.52 (m, 1H), 7.86-7.13 (m, 4 μl), 4.77 (m, 1H), 4.54-4.34 (m, 2H), 3.95 (2d, J=8.0 Hz, 1H), 3.75 (2d, J=18.4 Hz, 1H), 3.35-3.16 (2d, 6H), 2.78 (2dd, J=16.0, 4.4 Hz 1H), 2.54 (m, 1H), 2.39 (m, 1H), 2.35 (s, 1H), 1.06 (m, 6H)

### Preparation Example 3-1

### (4S,5S)-5-fluoromethyl-5-hydroxy-4-({[(5R)-5-isopropyl-3-(isoquinolin-1-yl)-4,5-dihydro-5-isoxazolyl]carbonyl}amino)-dihydrofuran-2-one (Compound I)

17.0 g (36.9 mmol) of the compound obtained from the above Preparation Example 2 (Compound V) and 6.6 mL (110 mmol) of acetic acid were dissolved in 123 mL (738 mmol) of 6 N hydrochloric acid aqueous solution, and stirred for about 4 hours. The inner temperature of the reaction mixture was adjusted to 0° C., and 150 mL of ethyl acetate was added thereto. 220 mL (660 mmol) of 3 N sodium hydroxide aqueous solution was slowly added to adjust the pH to about 3. The organic layer was separated, and the aqueous layer was extracted with ethyl acetate (150 mL×2). The combined organic phase was washed with 100 mL of salt water, and concentrated under reduced pressure. The residue was diluted with 50 mL of toluene, and concentrated again under reduced pressure to give a mixture of the compounds of Formula 6 (VI) and Formula 7 (VII) (15.4 g, quantitative yield, chemical purity: 87.0%).

¹H NMR (500 MHz, DMSO-d6): δ 8.99 (m, 1H), 8.65 (m, 1H), 8.19-7.78 (m, 4H), 5.15 (m, 1.5H), 4.77 (m, 1H), 4.42 (m, 0.5H), 3.91 (2d, J=17.6 Hz, 1H), 3.74 (m, 1H), 2.99 (m, 0.2H), 2.82 (m, 1H), 2.63 (m, 0.8H), 2.33 (m, 1H), 0.97 (m, 6H)

146 mL of toluene was added to 14.6 g (35.2 mmol) of the mixture of the compounds of Formula 6 and Formula 7 (chemical purity: 87.0%), and the mixture was heated up to 100° C. for complete dissolution. Then, 14 mg of the title compound (Compound I) was added thereto as a seed. The temperature was then slowly lowered to 20° C., and the reaction mixture was stirred to produce solid. 0.25 mL (1.8 mmol) of diisopropylamine (DIPA) was added thereto, and stirred at 20° C. for about 2 weeks. It was confirmed by HPLC that the ratio between the compound of Formula 6 (Compound VI) and the compound of Formula 7 (Compound VII) became 92.8:7.2. The reaction mixture was concentrated under reduced pressure to remove toluene. 88 mL of ethyl acetate was added thereto and the mixture was heated up to 65° C. for complete dissolution. Then, 88 mL of normal hexane was added thereto, and the temperature was slowly lowered and stirred at about 20° C. for 2 days. The resulting solid was filtered, and washed with a mixed solution of 15 mL of ethyl acetate and 15 mL of normal hexane. After drying the solid with nitrogen, the title compound (Compound I) was obtained as white solid with 54.7% of yield (8.0 g, chemical purity 98.6%) from the compound of Formula 2 (Compound II).

¹H NMR (CDCl₃): δ 9.02 (bs, 1H), 8.54 (d, J=5.5 Hz, 1H), 7.85 (d, J=7.95 Hz, 1H), 7.70 (m, 3H), 7.60 (bs, 1H), 4.86 (bs, 1H), 4.2-5.2 (bs, 2H), 4.05 (b, J=19.0 Hz, 1H), 3.78 (b, J=19.0 Hz, 1H), 2.7-3.1 (bm, 2H), 2.40 (m, 1H). 1.08 (dd, J=6.7, 4.9 Hz, 6H)

¹³C NMR (CDCl₃): 173.8, 172.4, 160.2, 147.6, 141.7, 136.8, 130.7, 129.0, 127.4, 127.3, 126.8, 122.9, 92.3, 82.7 (d, J=215 Hz), 48.9 (b), 44.6, 34.4, 33.9, 17.7, 16.3 Mass (ESI): 416.14 (M+1). [α]_{D}²⁵=+3.2 (c=1.0, acetonitrile)

### Preparation Example 3-2

### (4S,5S)-5-fluoromethyl-5-hydroxy-4-({[(5R)-5-isopropyl-3-(isoquinolin-1-yl)-4,5-dihydro-5-isoxazolyl]carbonyl}amino)-dihydrofuran-2-one (Compound I)

12.4 kg (26.86 mol) of the compound obtained from the above Preparation Example 2 (Compound V) and 4.9 kg (90.5 mol) of acetic acid were dissolved in 88.9 kg (52.9 kg of 35% hydrochloric acid+36.0 kg of water) of hydrochloric acid aqueous solution, and stirred for about 2 hours. The inner temperature of the reaction mixture was adjusted to 0° C., and 40.0 kg of ethyl acetate was added thereto. 161.0 kg (19.4 kg of sodium hydroxide+141.6 kg of water) of sodium hydroxide aqueous solution was slowly added to adjust the pH to about 3.5. The organic layer was separated, and the aqueous layer was extracted with ethyl acetate (30.0 kg×2). The combined organic phase was washed with 160.0 kg of water, and concentrated under reduced pressure. The residue was diluted with 73.0 kg of toluene, and concentrated again under reduced pressure. 97.0 kg of toluene and 0.1 kg (1.0 mol) of diisopropylamine were added to the resulting concentrated mixture (the mixture of the compounds of Formula 6 and Formula 7), and the mixture was heated up to 100° C. for complete dissolution. Then, 4.0 g of the object compound (Compound I) was added thereto as a seed. The temperature was slowly lowered to 40° C., and the reaction mixture was stirred to produce solid. The reaction mixture was stirred at about 40° C. for about 3 days. The resulting solid was filtered, and washed with 10.0 kg of toluene. After drying the solid with nitrogen, the title compound (Compound I) was obtained as light brown solid with 70.0% of yield (8.3 kg, chemical purity 94.9%) from the compound of Formula 2 (Compound II).

### Experimental Example 1

### Induction of Alcoholic Hepatitis

Male Sprague-Dawley rats (5 weeks of age, 170 g of body weight) were divided into four experimental groups: control diet group, control diet with CF1 group, ethanol (EtOH) diet group and EtOH diet with CF₁ group. Control diet group, control diet with CF1 group, EtOH diet group, and EtOH diet with CF₁ group were fed, respectively, with Lieber-Decarli control diet, Lieber-Decarli control diet and CF1, Lieber-Decarli liquid diet containing 6% ethanol, and Lieber-Decarli liquid diet containing 6% ethanol and CF1 for 12 weeks. CF1 was dissolved in PEG (polyethylene glycol) 400 and 5% ethanol solution (PEG 400:ethanol=2:1), and administered daily at 10 mg/kg by oral gavage. About 8 hours prior to rats' sacrifice, lipopolysaccharide (LPS) was intraperitoneally injected to increase the liver injury in the amount of 1 mg/kg.

### Experimental Example 2

### Measurement of Change of Body Weight and Liver Mass

After the 12 weeks' experiment, all rats were weighed prior to their sacrifice. After sacrifice, the livers were extracted and weighed. The results are shown in the following TABLE 1.

TABLE 1

| | Population | | |
|---|---|---|---|
| Group | (n) | Body Weight (g) | Liver Mass (g) |
| Control diet | 3 | 400.0 ± 00.00 | 10.67 ± 0.32 |
| Control diet with CF1 | 3 | 400.0 ± 20.00 | 10.37 ± 1.10 |
| EtOH diet | 5 | 366.0 ± 16.73 | 11.40 ± 1.02 |
| EtOH diet with CF1 | 5 | 372.5 ± 17.08 | 11.36 ± 0.87 |

As can be seen in the above TABLE 1, in EtOH diet group and EtOH diet with CF1 group, the body weights decreased slightly, while the liver masses increased slightly.

### Experimental Example 3

### Assay of Activity of Caspase-3 and Caspase-8 in Liver Tissue

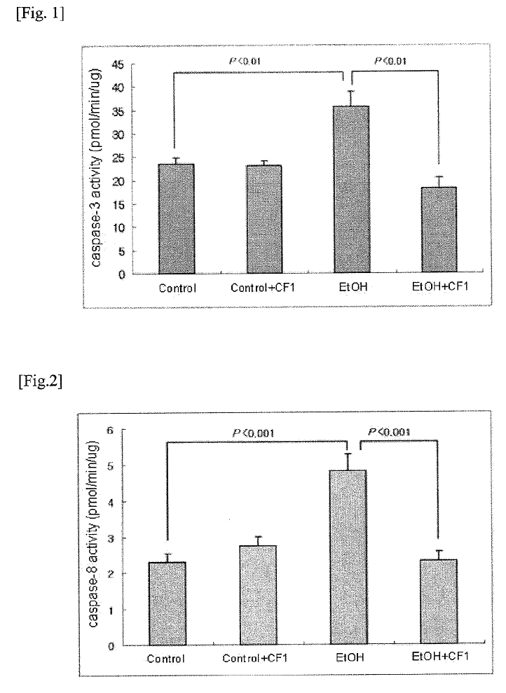

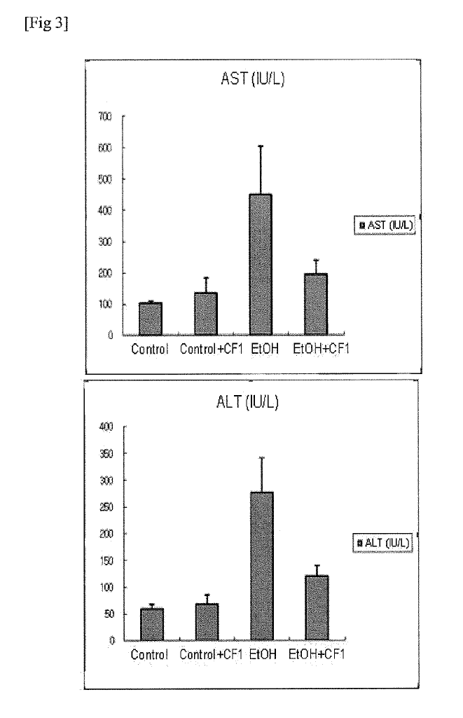

To investigate the role of caspase in alcohol-induced liver injury, the activities of caspase-3 and caspase-8 were assayed in cellular extraction of liver tissue. The activities of caspase-3 and caspase-8 were assayed by using EnzChek™ Caspase Assay Kit molecular Probes, USA) according to the manufacturer's instructions. The fluorometric assay results of caspase-3 and caspase-8 activities are shown in FIGS. 1 and 2, respectively.

As can be seen in FIGS. 1 and 2, when alcohol was ingested, activities of both caspase-3 and caspase-8 were significantly elevated compared with the control group. When CF1 which is a caspase inhibitor was administered, activities of both caspase-3 and caspase-8 were decreased to the level of control group.

### Experimental Example 4

### Determination of Serum Liver Enzymes in Association with Liver Injury

Liver injury was assessed by a biochemical assay of liver enzyme. After the 12 weeks' experiment, the blood samples were obtained from all rats' heart. Serum aspartate transaminase (AST) and alanine transaminase (ALT) values were analyzed for 100 μl of plasma from each blood sample with Hitachi 7050 automatic analyzer (Hitachi, Japan). The results are shown in FIG. 3.

As can be seen in FIG. 3, the control diet with CF1 group did not show significant change of AST and ALT values compared with the control diet group. The alcohol diet group showed elevated level of both AST and ALT as compared with the control diet group (AST 454±175 IU/L, ALT 336±163 IU/L). The alcohol diet with CF1 group showed significantly decreased level of both AST and ALT compared with the alcohol diet group (AST 194±22 IU/L, ALT 120±10 IU/L).

### Experimental Example 5

### Histological Assessment

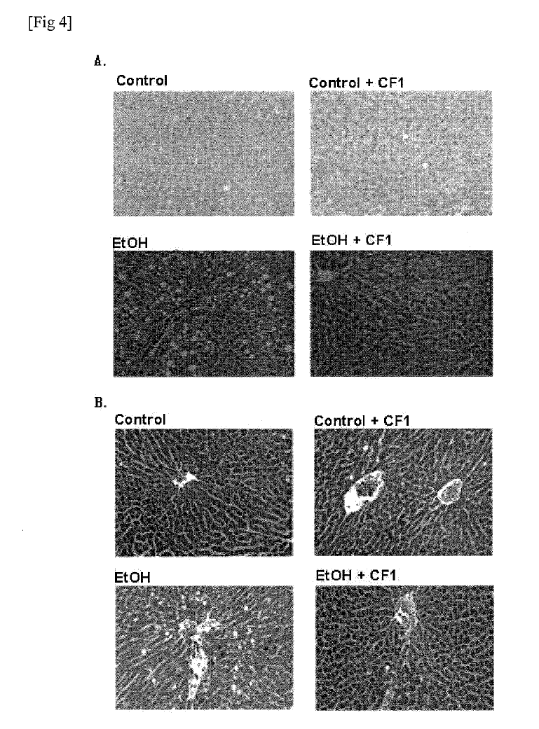

The extracted livers were fixed by 10% neutral formalin in PBS (phosphate buffered saline). Then, a paraffin section of 4 μm thickness was prepared, and stained with H&E (Hematoxylin & Eosin) and Masson's trichrome. The stained tissue was photographed at 100 times magnification (FIGS. 4A and 4B).

From FIG. 4A, it can be known that the alcohol diet group showed more fatty liver than the control diet group, and the administration of CF1 inhibited the increase of fatty liver. From FIG. 4B, it can be observed that the alcohol diet group and the alcohol diet with CF1 group showed some progression of hepatic fibrosis compared with the control diet group and the control diet with CF1 group, but there was no statistical significance.

### Experimental Example 6

### TUNEL Staining

To investigate apoptosis of liver cell according to alcohol ingestion, TUNEL staining was processed. TUNEL staining was carried out using in situ Cell Death Detection Kit (Roche Diagnostics, Germany) according to the manufacturer's instructions.

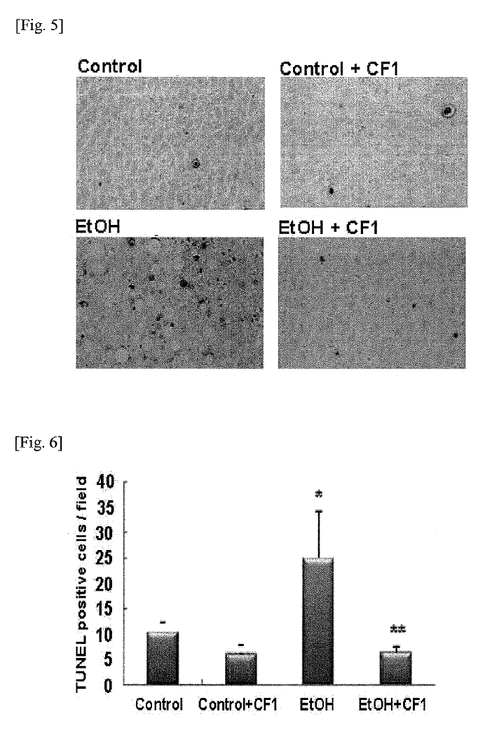

Stained tissue was photographed at 100 times magnification (FIG. 5). TUNEL positive cells per field were calculated. The results are shown in FIG. 6.

As can be seen in FIGS. 5 and 6, TUNEL positive cells were rarely observed in the control diet group and the control diet with CF1 group. However, the alcohol diet group showed statistically significant increase of TUNEL positive cells (alcohol diet group 25.14±9.14, control diet group 10.3±1.9 (unit: TUNEL positive cells/field)). TUNEL positive cells were rarely observed in the alcohol diet with CF1 group, like the control diet group (alcohol diet with CF1 group 6.4±1.1 TUNEL positive cells/field).

### INDUSTRIAL APPLICABILITY

CF1 of the present invention efficiently inhibits hepatocyte apoptosis via inhibition of caspase activity in alcohol-induced liver injury As a result, CF1 of the present invention also lowers levels of AST and ALT which are biochemical indicators of liver injury. Also, when CF1 is administered, decreases in fatty liver lesion can be observed by histological method, which means that CF1 can inhibit apoptosis efficiently. Therefore, CF1 of the present invention call be used as a therapeutic agent in treating alcohol-induced liver injury such as alcoholic hepatitis.

1. A pharmaceutical composition for treating alcohol-induced liver injury comprising (4S,5S)-5-fluoromethyl-5-hydroxy-4-({[(5R)-5-isopropyl-3-(isoquinolin-1-yl)-4,5-dihydro-5-isoxazolyl]carbonyl}amino)-dihydrofuran-2-one of Formula 1 or a pharmaceutically acceptable salt thereof:

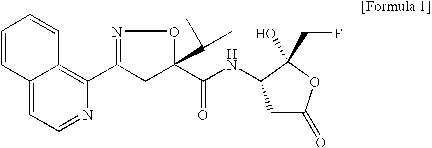

2. The pharmaceutical composition of claim 1, wherein the alcohol-induced liver injury is alcoholic hepatitis.

3. The pharmaceutical composition of claim 1, wherein a daily dose of (4S,5S)-5-fluoromethyl-5-hydroxy-4-({[(5R)-5-isopropyl-3-(isoquinolin-1-yl)-4,5-dihydro-5-isoxazolyl]carbonyl}amino)-dihydrofuran-2-one of Formula 1 is 0.1 to 100 mg per 1 kg of body weight.

4. The pharmaceutical composition of claim 1, which has a form of tablet or capsule.

5. A method for manufacturing of pharmaceutical composition for treating alcohol-induced liver injury comprising admixing (4S,5S)-5-fluoromethyl-5-hydroxy-4-({[(5R)-5-isopropyl-3-(isoquinolin-1-yl)-4,5-dihydro-5-isoxazolyl]carbonyl}amino)-dihydrofuran-2-one of Formula 1 or a pharmaceutically acceptable salt thereof with pharmaceutically acceptable carrier:

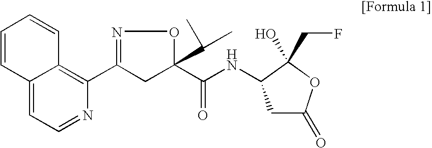

6. A use of (4S,5S)-5-fluoromethyl-5-hydroxy-4-({[(5R)-5-isopropyl-3-(isoquinolin-1-yl)-4,5-dihydro-5-isoxazolyl]carbonyl}amino)-dihydrofuran-2-one of Formula 1 or a pharmaceutically acceptable salt thereof for manufacturing a pharmaceutical composition for treating alcohol-induced liver injury:

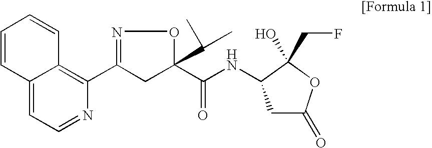

7. A method for treating alcohol-induced liver injury, comprising administrating an effective amount of the pharmaceutical composition of claim 1 to a patient suffering from alcohol-induced liver injury.

8. The method of claim 7, wherein a daily dose of 0.1 to 100 mg per 1 kg of body weight of (4S,5S)-5-fluoromethyl-5-hydroxy-4-({[(5R)-5-isopropyl-3-(isoquinolin-1-yl)-4,5-dihydro-5-isoxazolyl]carbonyl}amino)-dihydrofuran-2-one of Formula 1 is administered to the patient suffering from alcohol-induced liver injury.

9. A use of the pharmaceutical composition of claim 1 for treating alcohol-induced liver injury.
